(19) United States
(12) Patent Application Publication
(10) Pub. No.: US 2025/0213636 A1
(43) Pub. Date: Jul. 3, 2025

(54) A POLYHERBAL LOCAL ANAESTHETIC FORMULATION AND A METHOD OF PREPARATION

(71) Applicants: Hareendran Nair J, Kerala (IN); Shan SASIDHARAN, Kerala (IN)

(72) Inventors: Hareendran Nair J, Kerala (IN); Shan SASIDHARAN, Kerala (IN)

(21) Appl. No.: 18/853,025

(22) PCT Filed: Mar. 30, 2023
(86) PCT No.: PCT/IN2023/050312

§ 371 Date: Sep. 30, 2024

(30) Foreign Application Priority Data
Mar. 30, 2022 (IN) 202241018758

(51) Int. Cl. A61K 36/61; A61K 36/28; A61K 36/714; A61P 29/02

(52) U.S. Cl. CPC A61K 36/61; A61K 36/28; A61K 36/714; A61P 29/02; A61K 2236/15; A61K 2236/17; A61K 2236/33

(57) ABSTRACT

The invention disclosed herein describes a polyherbal local anaesthetic formulation and a method of preparation. The polyherbal anaesthetic formulation was formulated using the hydroalcoholic extract of herbs, viz., Syzygium aromaticum, Aconitum heterophyllum, Myristica fragrans, Aconitum ferox and Acmella ciliate along with the essential oil of Syzygium aromaticum and Nigella sativa. Alginate was used as the emulsifying agent (extracted from brown algae). The invention also discloses method of preparing the polyherbal formulation.

### FIELD OF INVENTION

The present invention relates to herbal anaesthetic agents. More specifically, the invention relates to polyherbal formulation used as a local or topical anaesthetic agent.

### BACKGROUND OF INVENTION

The invention disclosed herein describes the formulation, evaluation, clinical development and commercialization of a parenteral polyherbal local anaesthetic formulation to be included in the standard treatment protocol for local anaesthetic in minor surgeries and procedures. Before proceeding with the formulation of a local anaesthetic, we ensure that all the desired properties of an ideal local anaesthetic are met. An excellent local anaesthetic should have a rapid onset of action, which means the anaesthetic action should occur immediately after the administration without much delay. Another important property is that it should have a desired and comparably better duration of action, signifying that it should provide enough time for the successful completion of a procedure/surgery by the clinicians. For example, in preliminary research studies, clove is known for its anaesthetic property. The anaesthetic action of the clove will start spontaneously but will decline immediately from the body within a few minutes after administration. Due to this, clove/clove oil is not suitable for performing a procedure or surgery in the body. The next important property is the declining phase of anaesthetic potency, meaning that the action of an anaesthetic should decline slowly and steadily without producing any noxious effect on the patients. As we know, certain anaesthetic drugs will not normalize or rebalance polarisation and repolarization of the cells and this will cause several side effects, while also required acclimatisation time for the cells for a complete recovery from anaesthesia. Finally, it should have a broader therapeutic window. Unfortunately, none of the herbs used in the present formulation as a single drug didn't possesses the desired qualities of an ideal local anaesthetic. For this reason, the present polyherbal local anaesthetic dosage form was formulated in such a way that it could give all the desired pharmacological and pharmaceutical properties of an ideal local anaesthetic expected by the surgeon, patients, manufacturers, stockists, marketing professionals, etc.

The polyherbal anaesthetic formulation was formulated using the following medicinal herbs: Syzygium aromaticum, Aconitum heterophyllum, Myristica fragrans, Aconitum ferox, Acmella ciliate and Nigella sativa. The individual herbs selected for preparing the present formulation have been reported to have various pharmacological properties but doesn't have the potential to develop an ideal anaesthetic formulation with all the desired properties for a local anaesthetic. Therefore, the present dosage form was formulated using various combinations of the herbs mentioned above to get an ideal local anaesthetic agent.

The present formulation was formulated using the hydroalcoholic extract of herbs, viz., Syzygium aromaticum, Aconitum heterophyllum, Myristica fragrans, Aconitum ferox and Acmella ciliate along with the essential oil of Syzygium aromaticum and Nigella sativa. Alginate was used as the emulsifying agent (extracted from brown algae).

### SUMMARY OF INVENTION

The polyherbal formulation disclosed in this specification was formulated using the hydroalcoholic extract viz., of herbs, Syzygium aromaticum, Aconitum heterophyllum, Myristica fragrans, Aconitum ferox and Acmella ciliate along with the essential oil of Syzygium aromaticum and Nigella sativa. Alginate was used as the emulsifying agent (extracted from brown algae).

The herbal formulation comprises, Syzygium aromaticum extract of about 1-3%, Aconitum heterophyllum extract of about 0.2-1%, Myristica fragrans extract of about 1-3%, Aconitum ferox extract of about 0.2-1%, Acmella ciliate extract of about 1-3%, Syzygium aromaticum essential oil of about 0.2-1% and Nigella sativa essential oil of about 0.2-1%.

The specification also discloses a method of preparing hydroalcoholic extracts of Syzygium aromaticum, Aconitum heterophyllum, Myristica fragrans, Aconitum ferox and Acmella ciliate, comprising the following steps:
a) air-drying and coarsely powdering each of Syzygium aromaticum, Aconitum heterophyllum, Myristica fragrans, Aconitum ferox and Acmella ciliate separately;
b) macerating each of the Syzygium aromaticum, Aconitum heterophyllum, Myristica fragrans, Aconitum ferox and Acmella ciliate separately in 1:1 ratio of water and ethanol,
c) allow each of the macerate of step b) to stand for a period of 3 days at 55° C. with frequent agitation,
d) filtration of macerate of step b) to collect a filtrate,
e) remove ethanol from filtrate by methods such as vacuum evaporation using a rotary evaporator,
f) remove moisture content from filtrate of step e) using a vacuum oven at about 50-55° C. to obtain the dried residue;
g) sterilize the residue from step f) under UV light,
h) and store each extract of Syzygium aromaticum, Aconitum heterophyllum, Myristica fragrans, Aconitum ferox and Acmella ciliate for preparation of herbal formulation.

The specification further discloses a method of preparing herbal formulation stated above comprising steps of: mixing water phase, oil phase and emulsion agent. The step of preparing water phase filtrate further comprises steps of:
a) weighing extract of each of Syzygium aromaticum, Aconitum heterophyllum, Myristica fragrans, Aconitum ferox and Acmella ciliate as per the weight ratio provided in claim 1.
b) dissolving the extracts of step, a) in 50% volume of the final solution comprising sterile water,
c) homogenizing the extracts from step b) at about 3000-5000 rpm for 10-30 minutes,
d) the homogenized extracts of step c) is subjected to ultrasonication for 60-80 minutes:
e) filter the extract from step d) using a membrane filter of about 0.40 to 0.50 μm membrane filter;
f) filter the extract from step e) using a membrane filter of about 0.20 to 0.25 μm membrane filter;
g) steps c) to f) is repeated with undissolved substances from step f) in 30% volume of the final solution comprising sterile water,
h) dissolved filtrate obtained from steps f) and g) are pooled together: to obtain water phase filtrate of the herbal formulation.
i) The oil phase of the formulation comprises essential oils from Syzygium aromaticum and Nigella sativa, and the emulsifying the water phase and oil phase with alginate.

The specification also discloses a method of emulsification process comprises steps of:
a) taking the water phase extract in a homogenizer and homogenizing at about 600-800 rpm,
b) adding oil phase to the homogenized water phase component and homogenizing at about 1200-1600 rpm to obtain a primary emulsion,
c) adding 2% alginate solution from a stock solution of 0.5% concentration, to the primary emulsion, and
d) homogenizing the solution of step c) at about 1200-1600 rpm for about 10 minutes.

Following the preparation, the formulation was subjected to the In-Process Quality Control Tests (IPQC) and finished product QC. Subsequently, we have evaluated the skin irritation potential of this polyherbal anaesthetic formulation on a single application in Wistar albino rats to validate its safety in human trials. The rats were dermally exposed to the formulation and observed for 14 days. In the 14-day observation period, the formulation did not induce any reactions on intact skin sites following application. So, the primary skin irritation index of the polyherbal anaesthetic formulation was found to be 0.0, which appeared to fall under the category of non-irritant in rats. Hence, polyherbal anaesthetic formulations could be considered non-irritants and safe on rat skin. Based on these findings, the present formulations do not cause dermal irritation and skin sensitization toxicity and seems to be safe.

Consequently, we conducted the in-vivo analgesia duration test using electrical stimulation in wistar rats. The median duration of analgesia in rats administered with subcutaneous injection of polyherbal anaesthetic formulation and lignocaine 2% solution was 40 minutes and 35 minutes, respectively. The percentage of animals with analgesia was evaluated by measuring vocal response to the electrical stimulation and it showed a significant difference (p<0.05) among the test group and lignocaine (standard) group animals when compared with the animals in the normal control group. But there was no significant difference in vocalization response among the test group animals compared with standard drug-treated animals. The in-vivo analgesia duration test using electrical stimulation indicated that a single administration of the polyherbal anaesthetic formulation to rats provides significantly prolonged analgesia compared with the normal control group and standard drug.

The local anaesthetic effects of polyherbal-containing formulations were evaluated by the Tail Flick latency (TFL) test. From the results, it was clear that the polyherbal-containing formulations increased the TFL significantly. Polyherbal anaesthetic formulations recorded rapid maximum possible effect (MPE) in 10 minutes and lasted up to 50 minutes. These results clearly demonstrated a early onset of action, desirable duration of action and smooth recovery induced by the polyherbal anaesthetic formulation. Here in this study, polyherbal formulations recorded a pronounced anaesthetic effect that persisted for more than 50 minutes after the application, which signifies the excellent duration of action of the formulation. On the other hand, the lignocaine (standard drug) recorded activity only up to 30 minutes after application. The anaesthetic activity of polyherbal formulations and lignocaine revealed a more interesting and rapid anaesthetic effect in the first few minutes. But polyherbal formulations displayed constant platy activity for up to 50 minutes, which is superior to lignocaine. In addition to that, the activity of polyherbal anaesthetics withdraws immediately after 60 minutes, which shows fast and complete elimination of the drug.

Thus, the empirical evidences seemingly make us aware that the present local anaesthetic formulation has a significant onset of action in its application mode. Moreover, it also recorded a significant duration of action with a platy curve. In addition to this, the decline phase of the formulation is also fast. Further, it does not affect the cell and cellular functions, especially neurons permanently. In addition to that, this formulation does not induce any kinds of side effects or other allergic reactions. Thus, this formulation may be an ideal replacement for lignocaine, an existing standard of care in allopathic drugs.

### BRIEF DESCRIPTION OF DRAWINGS

The manner in which the present invention is formulated is given a more particular description below, briefly summarized above, may be had by reference to the components, some of which is illustrated in the appended drawing. It is to be noted: however, that the appended drawing illustrates only typical embodiments of this invention and are therefore, should not be considered limiting of its scope, for the system may admit to other equally effective embodiments.

The features and advantages of the present invention will become more apparent from the following detailed description a long with the accompanying figures, which forms a part of this application and in which:

FIG. 1: Dermal reaction scores (*T—Test, C—Control), the pictures (A) shows inflammation (edema and erythema) at 1 hr, (B) at 24 hr, (C) at 48 hrs, (D) at 72 hr, (E) at 7 days and (F) at 14 days.

FIG. 2: illustrates percentage (%) of animals with analgesia.

FIG. 3: In vivo TFL test for the evaluation of the local anaesthetic effects of LA-containing formulations.

### DETAILED DESCRIPTION OF INVENTION

The present invention will now be described more fully herein after. For the purposes of the following detailed description, it is to be understood that the invention may assume various alternative variations and step sequences, except where expressly specified to the contrary. Thus, before describing the present invention in detail, it is to be understood that this invention is not limited to particularly exemplified systems or embodiments that may of course, vary. The use of examples anywhere in this specification including examples of any terms discussed herein is illustrative only, and in no way limits the scope and meaning of the invention or of any exemplified term. Likewise, the invention is not limited to various embodiments given in this specification.

As used herein, the singular forms “a.” “an,” and “the” include plural reference unless the context clearly dictates otherwise. The term “and/or” means one or all of the listed elements or a combination of any two or more of the listed elements.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

When the term “about” is used in describing a value or an endpoint of a range, the disclosure should be understood to include both the specific value and endpoint referred to.

As used herein the terms “comprises”, “comprising”, “includes”, “including”, “containing”, “characterized by”, “having” or any other variation thereof, are intended to cover a non-exclusive inclusion.

Embodiments of the present invention will hereinafter be described in conjunction with the appended drawings provided to illustrate and not to limit the scope of the claims, wherein like designations denote like elements, and in which:

Results of the various literature searches suggest that herbal preparations and their associated phytochemicals have the potential to become local anaesthetic, general anaesthetic, antinociceptive, analgesic or sedative drugs. However, well-controlled clinical trials with phytochemical drug candidates and their practical applications to humans are still limited. Nevertheless, there is a possibility that selected phytochemicals could lead to anaesthetics and anaesthesia-related drugs. Terpenoids, alkaloids and flavonoids are expected to become novel anaesthetic agents of plant origin because they meet the mechanistic requirements to interact with receptors, channels and membranes and they have the characteristic molecular structures different from conventional drugs.

### Justification for Formulating the Polyherbal Anaesthetic

Local anaesthetic systemic toxicity (LAST) is a life-threatening adverse event that may occur after the administration of local anaesthetic drugs through a variety of routes. Increasing use of local anaesthetic techniques in various healthcare settings makes contemporary understanding of LAST highly relevant. Recent data have demonstrated that the underlying mechanisms of LAST are multifactorial, with diverse cellular effects in the central nervous system and cardiovascular system. Although neurological presentation is most common, LAST often presents atypically, and one-fifth of the reported cases present with isolated cardiovascular disturbance. Some of adverse effects of local anaesthetic include hypotension, nausea, postoperative pain, fever, vomiting, pruritus, back pain, headache, constipation, dizziness, and foetal distress. Due to the toxicities associated with local anaesthetic, there is urgent need of new local anaesthetic with fewer side effects.

### Example 1

### Selection of Herbs and Preparation of Extracts for Making the Formulation

### Selection of Herbal Material

The herbs, viz., Syzygium aromaticum, Aconitum heterophyllum, Myristica fragrans, Aconitum ferox and Acmella ciliate were selected for the present formulation. From Syzygium aromaticum, dried flower buds were used in preparing the extract for the anaesthetic formulation. From Aconitum heterophyllum and Aconitum ferox, dried tubers were used, from Myristica fragrans aril was used and from Acmella ciliate flower and arial parts were used in in preparing the extract for the anaesthetic formulation.

### Herbal Essential Oils

In addition to the above-mentioned herbs, we also used two essential oils for the preparation of the polyherbal anaesthetic formulation, i.e., the essential oil of Syzygium aromaticum and Nigella sativa.

### Collection and Authentication of Herb/Herbal Material

The herb materials selected for the present investigation, viz., Syzygium aromaticum, Aconitum heterophyllum, Myristica fragrans, Aconitum ferox and Acmella ciliate were collected from authorised dealers from Chithiramcode, Tamil Nadu and Tilak Bazar, Khari Baoli, Delhi.

### Extraction of Herbal Material

The air-dried, coarsely powdered Syzygium aromaticum, Aconitum heterophyllum, Myristica fragrans, Aconitum ferox and Acmella ciliate were separately subjected to maceration by placing in a stoppered macerating vessel with hydroalcoholic [water: ethanol (50:50)] as the extracting solvent and allowed to stand for a period of at least 3 days at 55° C. with frequent agitation until the soluble matter had dissolved. On the third day, it was filtered through eight layers of muslin cloth. After that, the filtrate was subjected to vacuum assisted evaporation using a rotary evaporator to remove the ethanol from the filtrate. Thus, the obtained filtrate was subjected to a vacuum oven at 50-55° C. to remove the water content and obtain the dried residue. All the extracts were sterilised under UV light. Finally, the extracts were subjected to sterility testing through microbiological analysis and in the end, solvent residue analysis to determine the presence of any residual solvents in the extract. Once all these processes were completed, the extracts were separately weighed to obtain the extractive yield and stored in airtight bottles at 4° C. for final formulation.

### Extraction of Essential Oils

Essential oil from Syzygium aromaticum was obtained using a steam distillation technique, whereas the oil from Nigella sativa was obtained by the cold press method. The oils thus obtained were subjected to autoclaving to remove the microbial contamination. Finally subjected to sterilisation by UV treatment and stored aseptically for final formulation.

### Emulsifying Agent

In this formulation, alginate (occurs naturally in brown algae) was used as an emulsifying agent. Alginate was subjected to sterilisation by UV treatment and stored aseptically for final formulation. The 2% alginate solution was taken from the 0.5% stock solution used in the formulation.

Concentration of each extracts and oils taken for final formulation

| S. No | Extract and oil | Concentration |
|---|---|---|
| 1 | Syzygium aromaticum extract | 2% |
| 2 | Aconitum heterophyllum extract | 0.5% |
| 3 | Myristica fragrans extract | 2% |
| 4 | Aconitum ferox extract | 0.5% |
| 5 | Acmella ciliate extract | 2% |
| 6 | Syzygium aromaticum essential oil | 0.5% |
| 7 | Nigella sativa essential oil | 0.5% |

### Example 2

### Formulation of Local Polyherbal Anaesthetic

The final formulation was strictly performed under an aseptic condition in a sterile cleanroom to avoid microbial contamination. First, each extract was carefully weighed according to the concentration of the formulation mentioned above. Then this was dissolved in half the quantity (i.e., if we wanted to prepare 100 ml of formulation, we should dissolve all the extract in 50 ml) of sterile water for injection. As we know, the extracts won't dissolve completely in the water. So, this was subjected to homogenization in a high-speed homogenizer at 5000 rpm for 15 min, followed by 3000 rpm for 15 min to dissolve the extracts completely in the water. Next, it was subjected to ultrasonication for 75 minutes. Thus, the obtained solution was subjected to membrane filtration first through a 0.45 μm membrane filter and then through a 0.22 μm membrane filter. After that, if any substance remains undissolved, we will continue the above-mentioned process, i.e. homogenization, ultrasonication and filtration in the remaining 30% sterile water for injection for maximum solubilization. Filtrates thus obtained are pooled together and this is labelled as the “water phase.” The oil phase for the formulation consists of sterile oils of Syzygium aromaticum and Nigella sativa. Finally, alginate was used as an emulsifying agent. Subsequently, all of these were subjected to emulsification using a high-speed homogenizer. During the emulsification process, the first water phase was added to the homogeniser and subjected to thorough mixing at 700 rpm. At that moment, the oil phase was added and allowed to mix completely with the water phase. Now the mixing speed has been increased to 1500 rpm. After some time, a primary emulsion will form. At this time, 2% alginate solution was taken from 0.5% stock solution and added to the primary emulsion. After that, the emulsification was continued at 1500 rpm to obtain an easily flowable, low-viscous, ashy white final product. In the final herbal composition, the ratio of water phase to oil phase to emulsion agent is 3:2:1 respectively.

This was again subjected to quality analysis, including microbiological and IPQC analysis. Then this was packed in single ampules (0.5 & 1 ml) for future use.

### (IPQC) Of Final Formulation

### 1. Physicochemical Properties

### Stability Checking

Stability is an essential quality attribute for drug products. The formulation was checked for their stability.

### Color Checking

The formulation was thoroughly checked for knowing any variation in color change.

### Odor Checking

The formulation was also checked for odor.

### Checking of pH

The formulation was also subjected to pH checking.

### 2. Leakage Test

A leakage test is employed to test the package integrity of the formulation. Package integrity reflects its ability to keep the product in and keep potential contamination out. It is because leakage occurs when a discontinuity exists in the wall of a package that can allow the passage of gas under pressure or concentration differential across the wall. Here, the leakage test of the formulation was done by the dye bath test.

### Dye Bath Test

The Dye Bath test is used for ampoules and vials to verify the leakage. The test container containing the formulation was immersed in a dye bath. Vacuum and pressure were applied for some time. After that, the container was removed from the dye bath and washed. The container was then inspected for the presence of dye, either visually or by means of UV spectroscopy. The dye may be blue, green, or yellowish-green in color. The dye test can be optimized by the use of a surfactant and or a low viscosity fluid in the dye solution to increase the capillary migration through the pores. The dye test is widely accepted in the industry and is approved for drug use. The test is inexpensive and requires no special equipment for visual dye detection. However, the test is qualitative, destructive and slow.

### 3. Particulate Matter in Injections

Particulate matter in injections and parenteral infusions consists of mobile undissolved particles, other than gas bubbles, unintentionally present in the solutions. For the determination of particulate matter, two procedures, Method 1 (Light Obscuration Particle Count Test) and Method 2 (Microscopic Particle Count Test), are specified hereinafter. Not all parenteral preparations can be examined for sub-visible particles by one or both of these methods. When Method 1 is not applicable, e.g. in case of preparations having reduced clarity or increased viscosity, the test should be carried out according to Method 2. Emulsions, colloids, and liposomal preparations are examples. Similarly, products that produce air or gas bubbles when drawn into the sensor may also require microscopic particle count testing. If the viscosity of the preparation to be tested is sufficiently high so as to preclude its examination by either test method, a quantitative dilution with an appropriate diluent may be made to decrease viscosity, as necessary, to allow the analysis to be performed. Mix the contents of the samples by slowly inverting the container 20 times successively. If necessary, cautiously remove the sealing closure. Clean the outer surfaces of the container opening using a jet of particle-free water and remove the closure, avoiding any contamination of the contents. For large-volume parenterals, single units are tested. For small-volume parenterals less than 25 mL in volume, the contents of 10 or more units is combined in a cleaned container: where justified and authorized, the test solution may be prepared by mixing the contents of a suitable number of vials and diluting to 25 mL with particle-free water or with an appropriate particle-free solvent when particle-free water is not suitable. Small volume parenterals having a volume of 25 mL or more may be tested individually. Powders for parenteral use are constituted with particle-free water or with an appropriate particle-free solvent when particle-free water is not suitable.

### 4. Isotonicity Checking

The tonicity of a solution may be defined as the characteristic represented by the effects that the solution has on the morphology of the cells that it comes into contact with. Isotonicity is important for parenteral preparations because the possibility that the product may penetrate red blood cells and cause hemolysis is greatly reduced if the solution is isotonic with blood i.e., the cells maintain their “tone”. Tonicity of our formulation was evaluated using egg membrane.

### 5. Osmolarity:

When the tonicity of a product is declared in its labeling, appropriate control of its osmolarity should be performed. Data generated during development and validation be sufficient to justify performance of this procedure as an in-process control, skip lot testing, or direct calculation of this attribute. The osmolarity of the formulation was measured using an osmometer. The osmolarity values of the formulation remained practically constant during the assessment period.

### 6. Pyrogen Test

### Limulus Amebocyte Lysate (LAL) Test

The LAL Assay is an in vitro assay used to detect the presence and concentration of bacterial endotoxins in drugs and biological products. Endotoxins, which are a type of pyrogen, are lipopolysaccharides present in the cell walls of gram-negative bacteria. Pyrogens as a class are fever-inducing substances that can be harmful or even fatal if administered to humans above specific concentrations. This test is based upon the gelling property of an enzyme, the limulus amebocyte lysate extracted from the horseshoe crab, limulus polyphormus. The enzyme gels in the presence of bacterial endotoxin and the degree of gelling is related to the amount of endotoxin present. A number of instruments are available for measuring the degree of gelling of enzymes. The test can quantify the amount of bacterial endotoxin present and provides better information regarding the quality of a product than the rabbit pyrogen test, which is more of a qualitative test. We have subjected the formulation to confirm the presence of pyrogen and results from experiments clearly point out that the extract is free from pyrogen.

### 7. Sterility Test

The sterility tests are intended to detect the presence of viable microorganisms in pharmaceutical preparations that are designed to be sterile. The test is based on the principle that if microorganisms are placed in a medium that provides optimum nutrition, moisture, PH, aeration, temperature, they can grow. Their presence will be indicated by the presence of turbidity in a clear medium. A test for sterility may be carried out by one of the following two methods:

### Membrane Filtration Technique

We subjected our formulation to the membrane filtration method to confirm the final sterility of the formulation. Use membrane filters having a nominal pore size of not greater than 0.45 μm whose effectiveness in retaining microorganisms has been established. Cellulose nitrate filters, for example, are used for aqueous, oily, and weakly alcoholic solutions, and cellulose acetate filters, for example, are used for strongly alcoholic solutions. Specially adapted filters may be needed for certain products (e.g., for antibiotics). The technique described below assumes that membranes about 50 mm in diameter will be used. If filters of a different diameter are used, the dilutions and the washings volumes should be adjusted accordingly.

Before testing, the filtration apparatus and membrane are sterilized by appropriate means. The apparatus is designed so that the solution to be examined can be introduced and filtered under aseptic conditions: it either permits the aseptic removal of the membrane for transfer to the medium or is suitable for carrying out the incubation after adding the medium to the apparatus itself. After filtration, the preparation membrane is cut into two halves. One halve was transferred to 100 ml of culture medium meant for the growth of the bacteria and incubated at 30 to 35° C. for not less than 3 days. The other halves are transferred to 100 ml of culture medium meant for fungi and incubated at 20-25° C. for not less than 7 days.

### 8. Content Uniformity and Weight

To determine the content of the active ingredient in each of the 10 vials that were taken at random. If the individual values obtained are all between 85 and 115 percent of the average value, the preparation under examination passes the test. If more than one individual value is outside the limits of 85 to 115 percent of the average value, or if any individual value is outside the limits of 75 to 125 percent of the average value, the preparation fails to comply with the test. If one individual value is outside the range of 85 to 115 percent but within the range of 75 to 125 percent of the average value, repeat the determination using another 20 randomly selected vials. The preparation under examination complies with the test if, in the total sample of 30 vials, not more than one individual value is outside the limits of 85 to 115 percent and none is outside the limits of 75 to 125 percent of the average value.

### 9. Extractable Volume

### Single Dose Containers

This method is used when the nominal volume does not exceed 5 mL. Use 6 vials, 5 for the tests and 1 for rinsing the syringe used. Using a syringe with appropriate capacity, rinse the syringe and withdraw as much as possible of the contents of one of the containers reserved for the test and transfer it, without emptying the needle, to a dry graduated cylinder of such capacity that the total combined volume to be measured occupies not less than 40% of the nominal volume of the cylinder. Repeat the procedure until the contents of the 5 containers have been transferred and measure the volume. The average content of the 5 containers is not less than the nominal volume and not more than 115% of the nominal volume. Alternatively, the volume of contents in milliliter can be calculated as mass in grams divided by the density.

### 10. Stability

To enhance the assurance of successful manufacturing operations, all process steps must be carefully reduced to writing after being shown to be effective. These process steps are often called standard operating procedures (SOPs). No extemporaneous changes are permitted to be made in these procedures: any change must go through the same approval steps as the original written SOP. Further external records must be kept to give assurance at the end of the production process that all steps have been performed as prescribed. Such in-process control is essential to assuring the quality of the product, since these assurances are even more significant than those from product release testing.

### Example 4

### Acute Dermal Irritation Study of Polyherbal Anaesthetic Formulation in Wistar Albino Rats

### Study Design

The test was carried out by employing OECD guideline 404. Approximately 24 h prior to the test, hair on the dorsal area of the trunk of the animals was removed by close clipping. Animals were treated in the following manner:

| Group | Study particulars | Dose |
|---|---|---|
| Group 1 | Initial | 1 animal |
| | test | 0.5 ml Polyherbal anaesthetic |
| | | formulation topically |
| Group 2 | Confirmatory | 2 animals |
| | test | 0.5 ml Polyherbal anaesthetic |
| | | formulation topically |

In the initial test, up to three test patches were applied sequentially to the animal. The first patch was removed after three minutes. A second patch was applied at a different site and removed after one hour. The observations at this stage indicated that exposure could humanely be allowed to extend to four hours, so a third patch was applied and removed after four hours, and the response was graded. The response was confirmed by using two additional animals, each with one patch, for an exposure period of four hours. At the end of the exposure period, which is normally 4 hours, the residual test chemical was removed, without altering the existing response or the integrity of the epidermis.

To determine the reversibility of the effects, the animals were observed for 14 days after the removal of the patches. All animals were examined for signs of erythema and oedema, and the responses were scored at 60 minutes, and then at 24, 48 and 72 hours after patch removal. For the initial test in one animal, the test site was also examined immediately after the patch had been removed.

Dermal reactions were graded and recorded according to the grades in the Table. 1

TABLE 1
Scoring system for skin reaction

| | |
|---|---|
| Reaction | Irritation score |

Erythema and Eschar Formation

| | |
|---|---|
| No erythema | 0 |
| Very slight erythema (barely perceptible) | 1 |
| Well defined erythema | 2 |
| Moderate to severe erythema | 3 |
| Severe erythema (beet redness) to eschar | 4 |
| formation preventing grading of erythema | |

Edema formation

| | |
|---|---|
| No edema | 0 |
| Very slight edema (barely perceptible) | 1 |
| Slight edema (edges of area well raised) | 2 |
| Moderate edema (raised approx. 1 mm) | 3 |
| Severe edema (raised more than 1 mm and | 4 |
| extending 4 beyond area of exposure) | |
| Maximal possible score for irritation | 8 |

*Other adverse changes in the skin sites shall be recorded and reported

### Administration of Test Item

Approximately 24 h prior to the test, fur was clipped on both sides of the spinal column (approximately 10 cm×15 cm). The test chemical to be tested was applied in a single dose to the skin of an experimental animal; untreated skin areas of the test animal served as the control. The test chemical was applied to a small area (approximately 6 cm²) of skin and covered with a gauze patch, which was held in place with non-irritating tape. The test chemical was first applied to the gauze patch, which was then applied to the skin. The patch was loosely held in contact with the skin by means of a suitable semi-occlusive dressing for the duration of the exposure period. The gauze patch was attached to the skin in such a manner that there was good contact and uniform distribution of the test chemical on the skin.

### Observations

The following observations were made during the study.

### Clinical Signs and Mortality

All animals were observed daily in the morning and afternoon for clinical signs and mortality during the study period.

There were no clinical signs of toxicity and mortalities in the animals tested. From the observation, it was clear that the test item had no adverse effect on the behavioural responses of the tested rats up to 14 days of observation (Table 2).

TABLE 2
Summary of clinical signs and mortality

| Animal no. | Sex | Clinical Signs | Mortality |
|---|---|---|---|
| 1 | Female | Nil | Nil |
| 2 | Female | Nil | Nil |
| 3 | Female | Nil | Nil |

### Body Weight, Food and Water Intake

The body weight of each rabbit was recorded prior to treatment on Day 1 and weekly thereafter up to the terminal day of study. The quantity of food and water consumed by rabbits in each cage was measured and recorded weekly from the day of the commencement of treatment.

In the present study, no treatment related changes in body weight were noted in animals (Table 3). No significant differences were noted in food and water consumption throughout the experiment (Table 4 & 5).

TABLE 3
Body weight and percentage weight gain in rats

| Animal | | Body weight (g) | | | Body weight gain (%) | |
|---|---|---|---|---|---|---|
| no. | Sex | Day 1 | Day 7 | Day 14 | 1-7 days | 1-14 days |
| 1 | Female | 205 | 210 | 210 | 2.44 | 2.44 |
| 2 | Female | 195 | 195 | 205 | 0.00 | 5.13 |
| 3 | Female | 195 | 200 | 205 | 2.56 | 5.13 |

TABLE 4
Mean food intake during treatment

| Animal | | Food intake (g) mean ± SD | | |
|---|---|---|---|---|
| no. | Sex | 1-7 days | 8-14 days | 1-14 days |
| 1 | Female | 18.57 ± 2.44 | 15.00 ± 4.08 | 16.79 ± 3.72 |
| 2 | Female | 16.43 ± 2.44 | 15.71 ± 3.45 | 16.07 ± 2.89 |
| 3 | Female | 17.14 ± 3.93 | 15.71 ± 3.45 | 16.43 ± 3.63 |

Values are expressed as mean ± SD

TABLE 5
Mean water intake during treatment

| | | Water intake (ml) mean ± SD | | |
|---|---|---|---|---|
| Group | Sex | 1-7 days | 8-14 days | 1-14 days |
| 1 | Female | 19.71 ± 5.22 | 16.14 ± 3.67 | 17.93 ± 4.71 |
| 2 | Female | 21.57 ± 5.03 | 17.43 ± 2.82 | 19.50 ± 4.47 |
| 3 | Female | 17.71 ± 5.06 | 20.29 ± 4.23 | 19.00 ± 4.67 |

Values are expressed as mean ± SD

### Skin Reactions

Dermal reactions were graded and recorded at 1, 24, 48 and 72 hours following test substance application according to the scoring system mentioned in Table 1. The dermal irritation scores were evaluated (as per Table 6) in conjunction with the nature and severity of lesions, and their reversibility or lack of reversibility.

TABLE 6
Primary or cumulative skin irritation index categories

| Mean Score | Response category |
|---|---|
| 0.0 to 0.4 | Negligible/Non-irritant |
| 0.5 to 1.9 | Slight |
| 2.0 to 4.9 | Moderate |
| 5.0 to 8.0 | Severe |

The dermal irritation scores are presented in Table 7. Any visible skin irritation and inflammation (edema and erythema) was not observed in rats treated with a single application of the polyherbal anaesthetic formulation during the study period as compared with control (FIG. 1). The combined irritation index score for acute single exposure dermal irritation was found to be ‘0’.

TABLE 7
Dermal reaction scores (*T—Test, C—Control)

| | | Rat Numbers | | | | | | | |
|---|---|---|---|---|---|---|---|---|---|
| | | 1 | | 2 | | 3 | | | |
| Time after treatment | | Female | | Female | | Female | | Mean score | |
| and Lesion Scores | | T | C | T | C | T | C | T | C |
| 1 | Hrs. | | | | | | | | |
| Erythema Score | | 0 | 0 | 0 | 0 | 0 | 0 | 0 | 0 |
| Edema Score | | 0 | 0 | 0 | 0 | 0 | 0 | | |
| 24 | Hrs. | | | | | | | | |
| Erythema Score | | 0 | 0 | 0 | 0 | 0 | 0 | 0 | 0 |
| Edema Score | | 0 | 0 | 0 | 0 | 0 | 0 | | |
| 48 | Hrs. | | | | | | | | |
| Erythema Score | | 0 | 0 | 0 | 0 | 0 | 0 | 0 | 0 |
| Edema Score | | 0 | 0 | 0 | 0 | 0 | 0 | | |
| 72 | Hrs. | | | | | | | | |
| Erythema Score | | 0 | 0 | 0 | 0 | 0 | 0 | 0 | 0 |
| Edema Score | | 0 | 0 | 0 | 0 | 0 | 0 | | |
| 7 | Days | | | | | | | | |
| Erythema Score | | 0 | 0 | 0 | 0 | 0 | 0 | 0 | 0 |
| Edema Score | | 0 | 0 | 0 | 0 | 0 | 0 | | |
| 14 | Days | | | | | | | | |

| | | | | | | | | |
|---|---|---|---|---|---|---|---|---|
| Erythema Score | 0 | 0 | 0 | 0 | 0 | 0 | 0 | 0 |
| Edema Score | 0 | 0 | 0 | 0 | 0 | 0 | | |

Primary Irritation Index = 0

### Example 5

### Evaluation of the Local Anaesthetic Activity of a Polyherbal Formulation in Wistar Albino Rats

### Electrical Stimulation Testing

In vivo analgesia duration test was conducted using electrical stimulation testing, which was used as a means of determining analgesia in animals and humans (Grant et al., 2000). Testing for analgesia was done by a vocal response to electrical stimulation (beginning at 1 mA and increasing to a maximum of 8 mA) at the skin directly overlying the abdomen at the site of injection using a current generator. The abdominal hair of the experimental rats was shaved off, and they were screened prior to injection to determine their vocalization threshold, the current required to produce a vocalization response.

Animals will be treated in the following manner.

| Groups | No. of Animals | Dose |
|---|---|---|
| Control | 6 | 0.9% Normal saline, 0.05 mL |
| | | subcutaneous route |
| Test | 6 | Polyherbal anaesthetic formulation, |
| | | 0.05 mL subcutaneous route |
| Standard | 6 | Local anaesthetic lignocaine 2% injection, |
| | | 0.05 mL subcutaneous route |

Polyherbal anaesthetic formulation, standard drug and 0.9% normal saline samples were injected subcutaneously into the abdomen of the experimental rats, followed by determination of analgesia at the fixed time intervals (5, 10, 20, 30, 40, 50, 60, and 70 min). If all the rats in one group did not vocalize to electrical stimulation 2 mA above threshold, it indicates 100% analgesia: if half of the rats in one group vocalized to electrical stimulation, it means 50% analgesia; and if all of the rats in one group vocalized to electrical stimulation, it indicates 0% analgesia (no analgesic effect).

### Result of Electrical Stimulation Testing

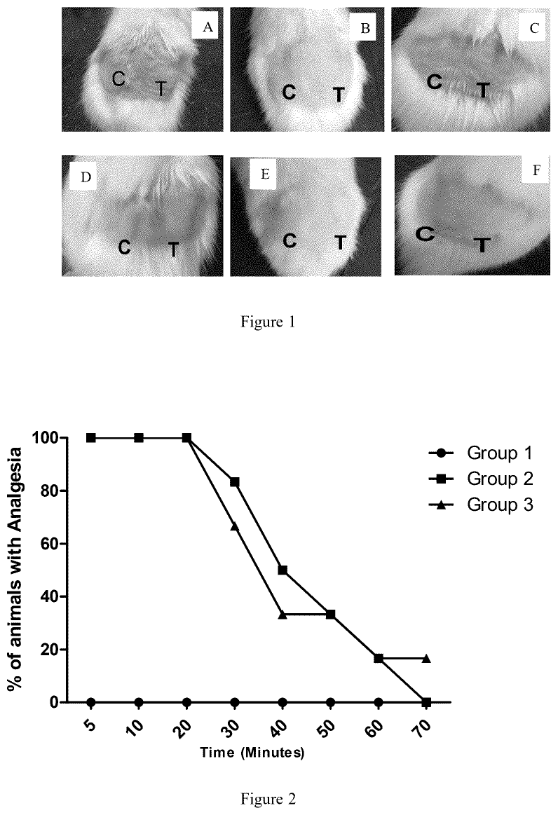

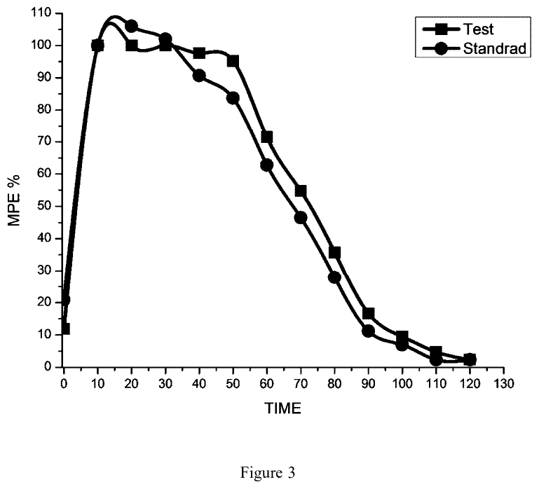

Findings from the in vivo analgesia duration test using electrical stimulation in rats are illustrated in FIG. 2. The figure indicates that a single administration of the particular Polyherbal anaesthetic formulation to rats provides significantly prolonged analgesia compared with the normal control group. The median duration of analgesia in rats administered with subcutaneous injection of Polyherbal anaesthetic formulation and lignocaine 2% solution was 40 minutes and 35 minutes, respectively. The percentage of animals with analgesia was evaluated by measuring vocal response to the electrical stimulation and it showed a significant difference (p<0.05) among the test group and lignocaine (standard) group animals when compared with the animals in the normal control group (Table 8). But there was no significant difference in vocalization response among the test group animals compared with standard drug-treated animals.

TABLE 8
Percentage (%) of animals with analgesia

| | Percentage (%) of animals with | | |
|---|---|---|---|
| Time intervals | analgesia in treatment groups | | |
| (minutes) | I | II | III |
| 5 | 0 | 100.00** | 100.00** |
| 10 | 0 | 100.00** | 100.00** |
| 20 | 0 | 100.00** | 100.00** |
| 30 | 0 | 83.33** | 66.67** |
| 40 | 0 | 50.00** | 33.33** |
| 50 | 0 | 33.33** | 33.33** |
| 60 | 0 | 16.67** | 16.67** |
| 70 | 0 | 0.00 | 16.67** |

### Tail-Flick Latency (TFL) Test

Local anaesthetic effects of LA-containing formulations were evaluated by tail-flick latency (TFL) test using a tail-flick measuring device (Ugo Basile, Varese, italy) (Puglia et al., 2011; Ouchi et al., 2013; Caffarel-Salvador et al., 2015). A noxious heat stimulus was applied via a focused, radiant heat light source to the dorsal surface of the tail. Tail-flick test started 5 min after the local application of different preparations, and the test was conducted every 10 min for 120 min. TFL was converted to represent the maximum possible effect (MPE) according to the following equation:

$$MPE ( \% ) = \frac{( Test latency - Baseline latency )}{( Cutoff time - Baseline latency )} \times 100$$

The baseline TFL was calculated as the mean of three different measurements taken at 10-min intervals. Baseline latencies were typically ranged from 2.5 to 3.0 s. A maximum cut-off latency of 10s was set to avoid tissue damage in analgesic animals. Results are expressed as mean±standard of eight rats per group.

### Result of Tail-Flick Latency (TFL) Test

The local anaesthetic effects of polyherbal-containing formulations were evaluated by the TFL test. As reported in FIG. 3, all the polyherbal-containing formulations increased the TFL significantly. There were no significant differences in baseline TFL between the saline control group and other formulations. The MPE in the saline group did not change during the experiment. Polyherbal formulations solution formulation recorded rapid MPE in 10 min and lasted up to 50 min. Thus, polyherbal formulations recorded a pronounced anaesthetic effect that persisted for more than 50 min after the application. The lignocaine, on the other hand, was only present for up to 30 minutes after application. The anaesthetic activity of both Polyherbal formulations and lignocaine revealed a more interesting and rapid anaesthetic effect in the first few minutes. But polyherbal formulations displayed constant activity for up to 50 min, which is superior to that of lignocaine.

It should be understood that the above examples described herein are for illustrative purposes only and that various modifications or changes in light if the specification will be suggestive to person skilled in the art and are to be included within the purview and scope of this application.

1. A polyherbal formulation for inducing anaesthesia comprising:
Syzygium aromaticum extract of about 1-3%, Aconitum heterophyllum extract of about 0.2-1%, Myristica fragrcins extract of about 1-3%, Aconitum ferox extract of about 0.2-1%, Acmella ciliate extract of about 1-3%, Syzygium aromaticum essential oil of about 0.2-1% and Nigella sativa essential oil of about 0.2-1%.

2. The formulation as claimed in claim 1 wherein the extracts of Syzygium aromaticum, Aconitum heterophyllum, Myristica fragrans, Aconitum ferox and Acmella ciliate is hydroalcoholic extract.

3. The formulation as claimed in claim 1, further comprising alginate as emulsifying agent.

4. The formulation as claimed in claim 3 wherein the alginate is extracted from brown algae.

5. A method of preparing hydroalcoholic extracts of Syzygium aromaticum, Aconitum heterophyllum, Myristica fragrans, Aconitum ferox and Acmella ciliate as claimed in claim 1, comprising steps of:
a) air-drying and coarsely powdering each of Syzygium aromaticum, Aconitum heterophyllum, Myristica fragrans, Aconitum ferox and Acmella ciliate separately;
b) macerating each of the Syzygium aromaticum, Aconitum heterophyllum, Myristica fragrans, Aconitum ferox and Acmella ciliate separately in 1:1 ratio of water and ethanol;
c) allow each of the macerate of step b) to stand for a period of 3 days at 55° C. with frequent agitation;
d) filtration of macerate of step b) to collect a filtrate;
c) remove ethanol from filtrate by methods such as vacuum evaporation using a rotary evaporator;
f) remove moisture content from filtrate of step e) using a vacuum oven at about 50-55° C. to obtain the dried residue;
g) sterilize the residue from step f) under UV light; and
h) store each extract of Syzygium aromaticum, Aconitum heterophyllum, Myristica fragrcins, Aconitum ferox and Acmella ciliate for preparation of herbal formulation of claim 1.

6. A method of preparing herbal formulation claimed in claim 1 comprising steps of:
mixing water phase, oil phase and emulsion agent in a ratio of 3:2:1 respectively;
wherein preparing water phase filtrate comprising steps of;
a) weighing extract of each of Syzygium aromaticum, Aconitum heterophyllum, Myristica fragrans, Aconitum ferox and Acmella ciliate as per the weight ratio provided in claim 1;
b) dissolving the extracts of step a) in 50% volume of the final solution comprising sterile water;
c) homogenizing the extracts from step b) at about 3000-5000 rpm for 10-30 minutes, d) the homogenized extracts of step c) is subjected to ultrasonication for 60-80 minutes;
e) filter the extract from step d) using a membrane filter of about 0.40 to 0.50 pm membrane filter;
f) filter the extract from step e) using a membrane filter of about 0.20 to 0.25 pm membrane filter;
g) steps c) to f) is repeated with undissolved substances from step f) in 30% volume of the final solution comprising sterile water,
h) dissolved filtrate obtained from steps f) and g) are pooled together; to obtain water phase filtrate of the herbal formulation, and oil phase comprising essential oils from Syzygium aromaticum and Nigella sativa; and emulsifying the water phase and oil phase with alginate.

7. The method as claimed in claim 6, wherein the emulsification process comprises steps of:
a) taking the water phase extract in a homogenizer and homogenizing at about 600-800 rpm;
b) adding oil phase to the homogenized water phase component and homogenizing at about 1200-1600 rpm to obtain a primary emulsion;
c) adding 2% alginate solution from a stock solution of 0.5% concentration, to the primary emulsion; and
d) homogenizing the solution of step c) at about 1200-1600 rpm for about 10 minutes;
to obtain the herbal composition.
